(19) United States
(12) Patent Application Publication
(10) Pub. No.: US 2025/0283032 A1
(43) Pub. Date: Sep. 11, 2025

(54) MAGNETIC CAPTURE DEVICE

(71) Applicant: LO BIOSCIENCES, INC., Rockville, MD (US)

(72) Inventor: Sasank VEMULAPATI, Silver Spring, MD (US)

(21) Appl. No.: 19/214,187

(22) Filed: May 21, 2025

Related U.S. Application Data
(63) Continuation of application No. PCT/US2023/080890, filed on Nov. 22, 2023.
(60) Provisional application No. 63/427,182, filed on Nov. 22, 2022.

(51) Int. Cl. C12N 5/00; G01N 1/34

(52) U.S. Cl. CPC C12N 5/0087; G01N 1/34

(57) ABSTRACT

Described herein are devices, systems, methods, and kits for use in the magnetic separation of biological entities from fluid samples.

### CROSS-REFERENCE

This application is a continuation of PCT/US2023/080890 filed Nov. 22, 2023, which claims benefit of U.S. Provisional Application No. 63/427,182 filed Nov. 22, 2022, which is incorporated herein by reference in its entirety.

### STATEMENT AS TO FEDERALLY SPONSORED RESEARCH

This present disclosure was made with government support under Small Business Grant Award ID 2111755 by the National Science Foundation. The government has certain rights in the disclosure.

### BACKGROUND

Microfluidics can separate biological entities from fluid samples. However, many approaches suffer from a lack of efficiency and simplicity for use in diagnostic and medical testing.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE DISCLOSURE

In some embodiments, disclosed herein is a method of capturing a target biological entity from a fluid sample, the method comprising: a. obtaining a package wherein the package contains a packed tube, wherein the packed tube is packed with: i. a plurality of magnetic beads functionalized to bind the target biological entity; and ii. an aggregation agent, wherein the aggregation agent aggregates the target biological entity when exposed to a plurality of the target biological entities; b. removing the packed tube from the package; c. disposing the fluid sample into the packed tube, wherein the fluid sample contains the target biological entity; d. after the disposing the fluid sample into the packed tube, capping the packed tube with a lid, wherein the lid comprises a dispensing tip; e. after the capping the packed tube with the lid, agitating the packed tube to mix the fluid sample with the plurality of magnetic beads and the aggregation agent; and f. after the agitating the packed tube, disposing the packed tube in proximity of a magnetic field, wherein the magnetic field separates the magnetic beads from a supernatant.

In some embodiments, the packed tube comprises an interior surface, wherein the interior surface of the packed tube is coated at least in part with a chelation agent.

In some embodiments, the packed tube contains a chelation agent.

In some embodiments, the packed tube contains a calcium chelator.

In some embodiments, the fluid sample is mixed with a chelation agent prior to disposing the fluid sample into the packed tube.

In some embodiments, the method further comprises mixing the fluid sample with a calcium chelator prior to disposing the fluid sample into the packed tube.

In some embodiments, the method further comprises disposing a chelation agent into the packed tube after removing the packed tube from the package and before capping the packed tube with the lid.

In some embodiments, the method further comprises: g. dispensing the supernatant through the dispensing tip into a collection tube.

In some embodiments, the method further comprises: f. squeezing the packed tube to dispense the supernatant.

In some embodiments, the packed tube comprises an interior surface, wherein the interior surface of the packed tube is coated at least in part with an anticoagulant.

In some embodiments, the packed tube contains an anticoagulant.

In some embodiments, the fluid sample is mixed with an anticoagulant prior to disposing the fluid sample into the packed tube.

In some embodiments, the method further comprises mixing the fluid sample with a chelation agent prior to disposing the fluid sample into the packed tube.

In some embodiments, the method further comprises disposing an anticoagulant into the packed tube after removing the packed tube from the package and before capping the packed tube with the lid.

In some embodiments, the fluid sample is blood.

In some embodiments, the target biological entity is a blood cell.

In some embodiments, the packed tube is made of a deformable material.

In some embodiments, the packed tube is coated with a hydrophobic material.

In some embodiments, the hydrophobic material comprises a fluoropolymer.

In some embodiments, the plurality of magnetic beads has a mean greatest diameter of no larger than 4 μm.

In some embodiments, the aggregation agent is an antibody.

In some embodiments, the aggregation agent is present at a concentration from about 3 mg/mL to about 5 mg/mL in water.

In some embodiments, the plurality of magnetic beads and the aggregation agent are lyophilized.

In some embodiments, the method further comprises lyophilizing the plurality of magnetic beads and the aggregation agent prior to disposing the fluid sample into the packed tube.

In some embodiments, the supernatant is substantially free of the target biological entity.

In some embodiments, the lid further comprises a size-exclusion membrane in fluid communication with the dispensing tip.

In some embodiments, the lid further comprises a filter in fluid communication with the dispensing tip.

In some embodiments, the magnetic beads are substantially separated from the supernatant within one minute of disposing the packed tube in proximity of the magnetic field.

In some embodiments, at least about 95% of the target biological entity is separated from the supernatant.

In some embodiments, at least about 99% of the target biological entity is separated from the supernatant.

A kit comprising: a packed tube, wherein the packed tube is packed with i. a plurality of magnetic beads functionalized to bind a target biological entity; and ii. an aggregation agent, wherein the packed tube is suitable to contain a fluid sample; a lid suitable for capping the packed tube, wherein the lid comprises a dispensing tip; and a magnet, wherein the packed tube, the lid, and the magnet are packaged within a common packaging.

In some embodiments, the kit further comprises a filter paper suitable to trap a residue from a supernatant.

In some embodiments, the magnet is disposed in a bracket, wherein the bracket comprises a receptacle suitable for holding the packed tube in proximity to the magnet.

In some embodiments, the magnet is a neodymium magnet.

In some embodiments, the kit further comprises a chelation agent within the common packaging.

In some embodiments, the kit further comprises a calcium chelator within the common packaging.

In some embodiments, the kit further comprises an anticoagulant within the common packaging.

In some embodiments, the packed tube comprises an interior surface, wherein the interior surface of the packed tube is coated at least in part with a chelation agent.

In some embodiments, the packed tube comprises an interior surface, wherein the interior surface of the packed tube is coated at least in part with an anticoagulant.

In some embodiments, the packed tube is made of a deformable material.

In some embodiments, the packed tube is coated with a hydrophobic material.

In some embodiments, the hydrophobic material comprises a fluoropolymer.

In some embodiments, the plurality of magnetic beads has a mean greatest diameter of no larger than 4 μm.

In some embodiments, the aggregation agent is an antibody.

In some embodiments, the plurality of magnetic beads and the aggregation agent are lyophilized.

In some embodiments, the lid further comprises a size-exclusion membrane in fluid communication with the dispensing tip.

In some embodiments, the lid further comprises a filter in fluid communication with the dispensing tip.

### BRIEF DESCRIPTION OF THE DRAWINGS

FIG. 1 illustrates an example of a magnetic bead-based capture device disclosed herein, wherein the device comprises a tube with: (a) a thixotropic gel inside the tube; (b) a magnet inside the tube, wherein the magnet is disposed below the thixotropic gel; and (c) a piezoelectric buzzer at the bottom of the tube.

FIG. 2 illustrates an example of a magnetic bead-based capture device disclosed herein, wherein the device comprises: (a) a fluidic serpentine channel with positive or negative pressure; (b) a magnetic field near the end of the fluidic serpentine channel; and (c) an outlet at the end of the channel that collects biological material.

FIG. 3 illustrates an example of a magnetic bead-based capture device disclosed herein, wherein the device comprises: (a) a plastic, disposable housing unit with a thumb-drive that has a dried plasma spot collection membrane and can be inserted into the base of the housing unit; and (b) a collection tube that can fit into the housing unit; and (c) a source of magnetic field in proximity to the tube.

FIG. 4A and FIG. 4B illustrate an example of a magnetic bead-based capture device disclosed herein and a method of use, wherein the device comprises: (a) a tube; (b) a tube lid with a small hole; (c) a narrow cylinder that can be inserted into the lid hole; and (d) a source of a magnetic field in proximity to the tube. FIG. 4A illustrates an example where the supernatant can be dispensed from the top of the tube. FIG. 4B illustrates an example where the supernatant can be dispensed from the bottom of the tube.

FIG. 5 illustrates an example of a magnetic bead-based capture device disclosed herein, wherein the device comprises: (a) a tube or vessel with a wide base that has a high surface area to volume ratio; (b) a magnet at the base of the tube; and (c) a tube lid with an opening that dispenses the biological material or supernatant.

FIG. 6A and FIG. 6B illustrate examples of a magnetic bead-based capture device disclosed herein, wherein the device comprises: (a) a larger compartment in which magnetic beads, aggregation agent, and sample fluid are mixed; and (b) a smaller compartment in which magnetic beads and captured entities are collected, wherein (a) and (b) are connected by a thin, horizontal connection tube that can be interrupted by a physical pin to create a barrier between the two compartments; and (c) a source of a magnetic field. FIG. 6A illustrates an example where the source of a magnetic field can be placed on the smaller compartment. FIG. 6B illustrates an example where the source of a magnetic field can be placed on the connection tube.

FIG. 7A, FIG. 7B, and FIG. 7C illustrate an example of a magnetic bead-based capture device disclosed herein, wherein the device comprises: (a) a tube; (b) a housing unit with a mechanical assembly configured to contain the tube, and (c) a magnet at the bottom of the housing unit. FIG. 7A illustrates an example where the housing unit can have two buttons on the sides. FIG. 7B and FIG. 7C illustrate examples where the housing unit can have a threaded screw assembly.

FIG. 8A and FIG. 8B illustrate an example of a magnetic bead-based capture device disclosed herein, wherein the device comprises: (a) a tube; (b) a spring-loaded cap attached to an opening of the tube; and (c) a magnet at the bottom of the tube. FIG. 8A illustrates an example where a rod with a flexible air-filled bulb can be inserted into the spring-loaded tube cap. FIG. 8B illustrates an example where the air-filled bulb and spring are compressed.

FIG. 9 illustrates an example of a magnetic bead-based capture device disclosed herein, wherein the device comprises: a serpentine channel tube, housed in a unit that has push buttons, each push button configured to release a corresponding reagent (e.g., aggregate agent, antibodies).

FIG. 10 illustrates an example of a magnetic bead-based capture device disclosed herein, wherein the device comprises: a finger sleeve with an opening for needle prick and collection of blood.

FIG. 11 illustrates an example of a magnetic bead-based capture device disclosed herein, wherein the device comprises: (a) a tube; (b) a lid to contain a fluid sample; and (c) a source of a magnetic field in proximity to the tube.

FIG. 12 illustrates an example of a magnetic bead-based capture device disclosed herein, wherein the device comprises: (a) a tube; (b) a tube lid with a dispensing tip; (c) a tube holder with buttons that can be depressed to move in the radial direction and translated along axial direction; and (d) a source of a magnetic field at the bottom of the magnetic holder.

FIG. 13 illustrates an example of a magnetic bead-based capture device disclosed herein, wherein the device comprises: (a) a collection tube to receive the sample; (b) a tube holder; and (c) a storage tube.

FIG. 14 shows the percentage of plasma yield using 3 mg/mL, 4 mg/mL, or 5 mg/mL aggregation agent in water or water with buffering salts at 1 minute, 2 minutes, or 4 minutes incubation time with the magnetic bead.

### DETAILED DESCRIPTION

### Magnetic Bead-Based Capture Devices

Disclosed herein are devices relating to the magnetic bead-based capture of biological material. A device can comprise, for example: (a) a compartment (e.g., tube or vessel or channel) that houses a plurality of magnetic beads, aggregation agent, and sample fluid; and (b) a magnet and/or magnetic field that attracts the magnetic beads. In some embodiments, the compartment (e.g., tube or vessel or channel) can a packed compartment with plurality of magnetic beads and aggregation agent.

### Device Using Piezoelectric Buzzer

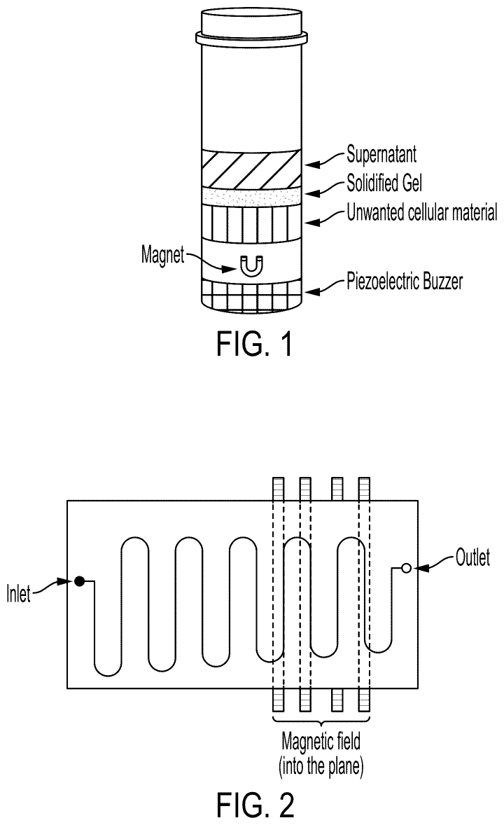

A non-limiting example of a device comprises a tube with: (a) a thixotropic gel inside the tube, (b) a magnet inside the tube, wherein the magnet is disposed below the thixotropic gel, and (c) a piezoelectric buzzer at the bottom of the tube (FIG. 1). The thixotropic gel can have a specific gravity that is greater than that of the supernatant (e.g., serum or plasma), but have a specific gravity lower than that of blood cells (e.g., red blood cells, white blood cells, and platelets). In some embodiments, the thixotropic gel can be a hydrophobic gel with polyester based formulation or any other formulation of thixotropic gel. In some embodiments, the thixotropic gel can be replaced with a mixture of silicon fluid and a hydrophobic, powdered silica or a mixture of a hydrocarbon polymer and a powered silica. In some embodiments, the thixotropic gel is a combination of silica polymer or silica oil as a base with Dicumyl Peroxide added as a crosslinker. In some embodiments, a magnetic mesh can be used in place of a thixotropic gel to separate the sample fluid from biological entities. In some embodiments, the magnetic mesh can be oriented by the magnet placed underneath to prevent the mixing of supernatant with captured biological entities. In some embodiments, a combination of a magnetic mesh and a thixotropic gel can be used to separate the fluid sample from the biological entities. In some embodiments, the thixotropic gel contains magnetic particles and is magnetically responsive. In some embodiments, the magnetic particles are micro-scale iron filings of various sizes that are impregnated within the thixotropic gel during production to induce a magnetic responsiveness. In some embodiments, the magnetic particles in the gel are macro-sized iron oxide particles or shavings. In some embodiments, the magnetic gel is dispensed on top of the beads and aggregation agent and is displaced by the blood cells.

The piezoelectric buzzer at the bottom of the device can be turned on and off. Turning on the piezoelectric buzzer can create enough agitation to mix the fluid sample and the plurality of magnetic beads in the tube, to overcome the magnetic force from the magnet, and to liquify the thixotropic gel. The piezoelectric buzzer can be turned off to stop the mixing of fluid sample and magnetic beads, and to resolidify the thixotropic gel. In some embodiments, the piezoelectric buzzer contains a source of a magnetic field that can be used to manipulate the magnetically responsive gel.

### Device Using Fluidic Serpentine Channel

A device can comprise, for example: (a) a fluidic serpentine channel with positive or negative pressure; (b) a magnetic field near the end of the fluidic serpentine channel; and (c) an outlet at the end of the channel that collects biological material (FIG. 2).

The fluidic serpentine channel can have multiple loops to allow complete mixing of fluid sample and magnetic beads. In some embodiments, the fluidic serpentine channel can have immobilized antibodies on the internal surface of the channel. In some embodiments, the positive or negative pressure applied to the channel can extract the supernatant from the captured biological material after mixing of fluid sample and magnetic beads and exposure to magnetic field. In some embodiments, the fluidic serpentine channel can be a disposable card that automatically collects and separates the sample fluid.

In some embodiments, the magnetic field can be generated by a single rare earth magnet or electromagnet. In some embodiments, the magnetic field can be a neodymium magnet. The magnetic field can be disposed perpendicularly, into the cross-sectional plane of the fluidic serpentine channel near the end of the channel. In some embodiments, the magnetic field can capture cells of different sizes by inducing fluctuations in the magnetic field.

### Device Using Plastic Disposable Housing Unit

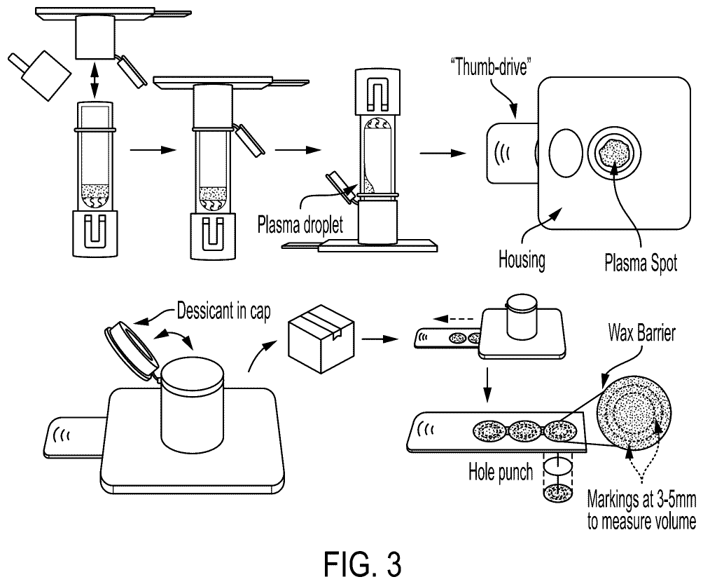

A device can comprise, for example: (a) a plastic, disposable housing unit with a thumb-drive that has a dried plasma spot collection membrane and can be inserted into the base of the housing unit; and (b) a collection tube that can fit into the housing unit; (c) a source of magnetic field in proximity to the tube (FIG. 3).

In some embodiments, the thumb-drive can have three separate spot collection membranes, wherein the membranes are connected by chromatography paper with wax barriers. Each spot collection membranes can have graduated markers at different radii (e.g., about 3 mm, about 4 mm, or about 5 mm), wherein the graduated markings can further control the output volume. In some embodiments, the thumb-drive further comprises additional drying spots. In some embodiments, the third spot collection can be used to collect overflow.

Non-limiting examples of a collection tube include portable capillary blood collection systems, lancets, venipuncture, and capillary tubes.

In some embodiments, the magnetic field can be generated by a single rare earth magnet or electromagnet or any variations thereof. In some embodiments, the magnetic field can be a neodymium magnet.

### Device Using Tube That Can Directly Collect Fluid Sample

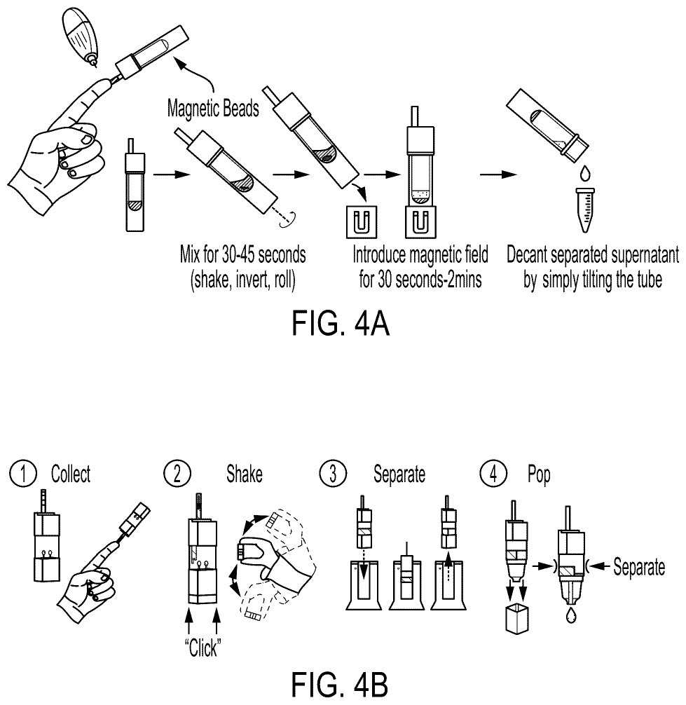

A device can comprise, for example: (a) a tube; (b) a tube lid with a small hole; (c) a narrow cylinder that can be inserted into the lid hole; and (d) a source of a magnetic field in proximity to the tube (FIG. 4A and FIG. 4B).

In some embodiments, the tube can be at least about 40 mm, at least about 45 mm, or at least about 50 mm in length. The diameter can be at least about 13 mm, at least about 14 mm, at least about 15 mm, at least about 16 mm, or at least about 17 mm in diameter.

The tube can hold the sample fluid, magnetic beads, and aggregation agent. In some embodiments, the tube is a packed tube comprising the magnetic beads and aggregation agent. In some embodiments, the magnetic beads and aggregation agent are lyophilized. In some embodiments, the tube is a packed tube comprising lyophilized magnetic beads and aggregation agent. In some embodiments, the tube can have one opening at the top of the tube to decant the supernatant. In some embodiments, the tube can have another opening at the bottom of the tube, wherein a bottom lid can be placed to expose or close the opening. In some embodiments, the tube can be made from a flexible material (e.g., plastic), wherein the tube can be squeezed to dispense supernatant from the bottom opening of the tube. In some embodiments, the tube can have an inner wall that is functionalized with a hydrophobic coating (e.g., silica, Teflon, fluoropolymers). In some embodiments, the tube can have an inner wall that is functionalized to be non-wettable.

In some embodiments, the magnetic field can be generated by a single rare earth magnet or electromagnet or any variations thereof. The single rare earth magnet or electromagnet can be in the shape of a tube holder to hold the tube in place and attract magnetic beads. In some embodiments, the magnetic field can be a neodymium magnet.

### Device Using High Surface Magnetic Base

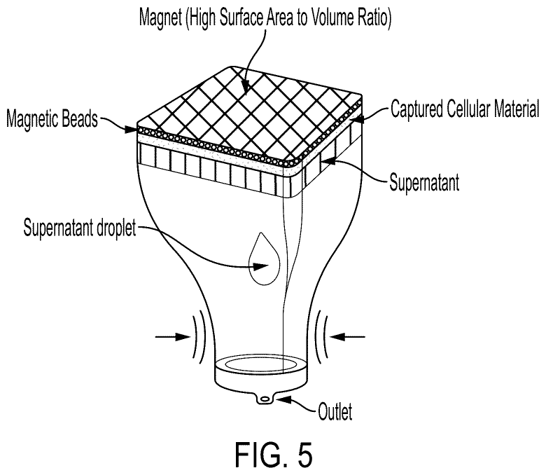

A device can comprise, for example: (a) a tube or vessel with a wide base that has a high surface area to volume ratio; (b) a magnet at the base of the tube; and (c) a tube lid with an opening that dispenses the biological material or supernatant (FIG. 5). In some embodiments, the tube or vessel can be of flexible material (e.g., plastic), wherein the material can be pinched to dispense resulting supernatant.

### Device Using Two Compartments

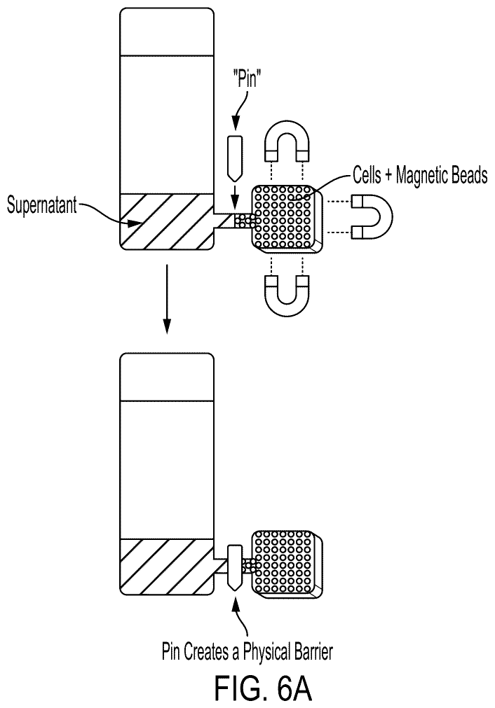

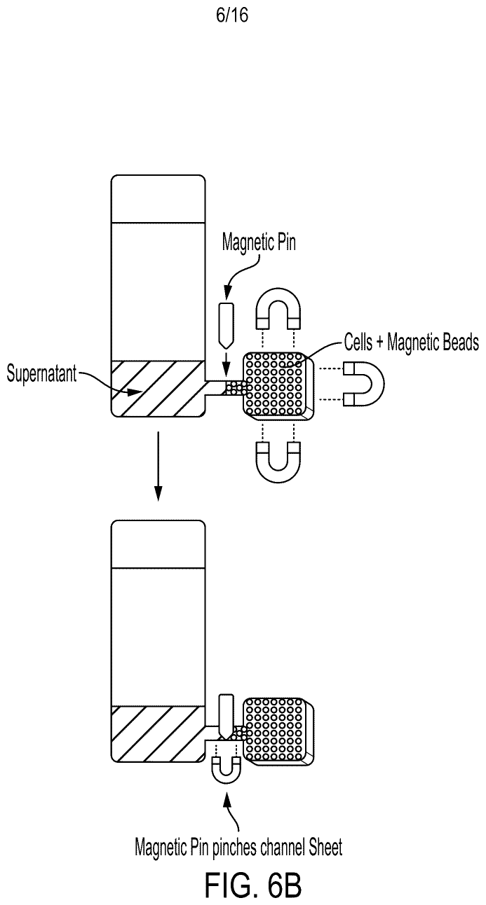

A device can comprise, for example: (a) a larger compartment in which magnetic beads, aggregation agent, and sample fluid are mixed; and (b) a smaller compartment in which magnetic beads and captured entities are collected, wherein (a) and (b) are connected by a thin, horizontal connection tube that can be interrupted by a physical pin to create a barrier between the two compartments; and (c) a source of a magnetic field (FIG. 6A and FIG. 6B).

In some embodiments, the large compartment is a packed compartment comprising the magnetic beads and aggregation agent. In some embodiments, the magnetic beads and aggregation agent are lyophilized. In some embodiments, the large compartment is a packed compartment comprising lyophilized magnetic beads and aggregation agent. In some embodiments, the two compartments can have separate openings, wherein the contents of the two compartments can be emptied separately.

In some embodiments, the physical pin can be sharp on one end, and the rest of the pin can be made from a flexible gasket material to create a seal between the two compartments.

The magnetic field can be introduced to the smaller compartment to attract magnetic beads and entities attached to the magnetic beads. In some embodiments, the magnetic field can be generated by a single rare earth magnet or electromagnet or any variations thereof. In some embodiments, the magnetic field can be a neodymium magnet.

In some embodiments, the physical pin can be magnetic, wherein the magnetic field can be introduced to the magnetic pin. Introduction of the magnetic field to the magnetic pin can pinch the channel connecting the two compartments to create a physical barrier, wherein the channel is made of flexible material (e.g., plastic).

### Device Using Toggling Between Two States

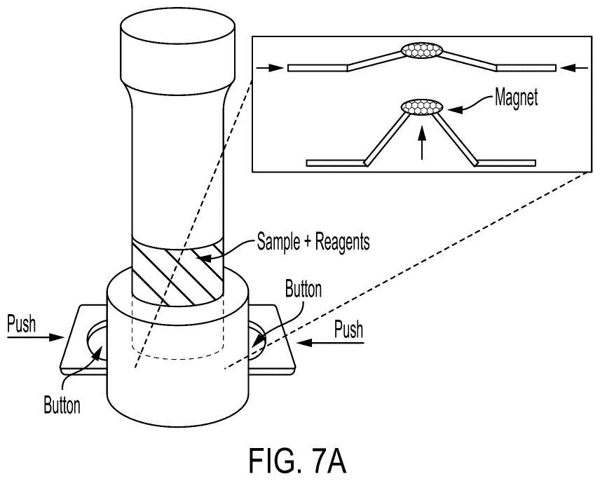

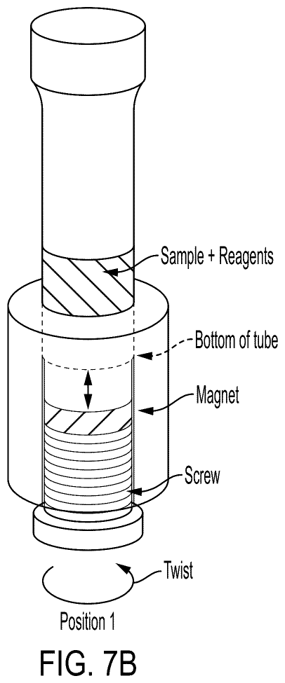

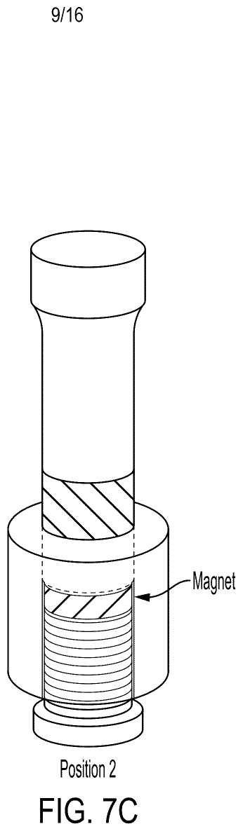

A device can comprise, for example: (a) a tube; (b) a housing unit with a mechanical assembly configured to contain the tube, and (c) a magnet at the bottom of the housing unit. (FIG. 7A, FIG. 7B, FIG. 7C).

The housing unit can have two buttons on the sides. In some embodiments, the two buttons can be connected to a linkage system, wherein the act of pressing the buttons can bring a magnet into contact with the bottom of the tube. The housing unit can have a threaded screw assembly, wherein a screw can be turned or twisted into the threaded screw assembly to bring the magnet into contact with the bottom of the tube. In some embodiments, the distance between the bottom of the tube and the magnet can be 4 mm, 5 mm, 6 mm, 7 mm, 8 mm, 9 mm, or 10 mm.

The tube can hold the sample fluid, aggregation agent, magnetic beads and can be placed in the housing unit for separation of biological entities.

### Device Using Spring Loaded Cap

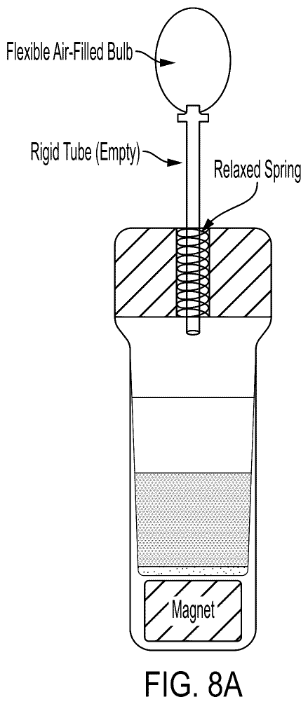

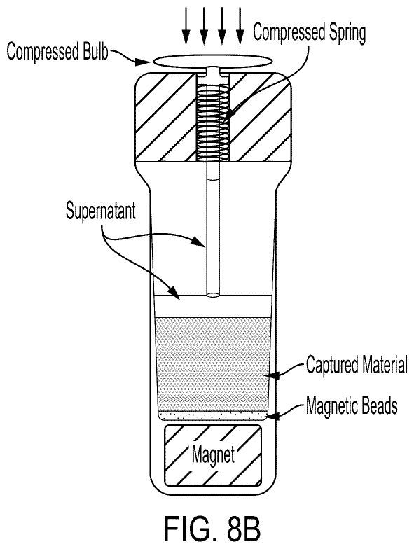

A device can comprise, for example: (a) a tube; (b) a spring-loaded cap attached to an opening of the tube; and (c) a magnet at the bottom of the tube (FIG. 8A and FIG. 8B).

The tube can hold the sample fluid, magnetic beads, and aggregation agent. In some embodiments, a rigid, hollow rod with a flexible air-filled bulb can be inserted into the spring-loaded tube cap to draw up the resulting supernatant.

### Device With Push Buttons

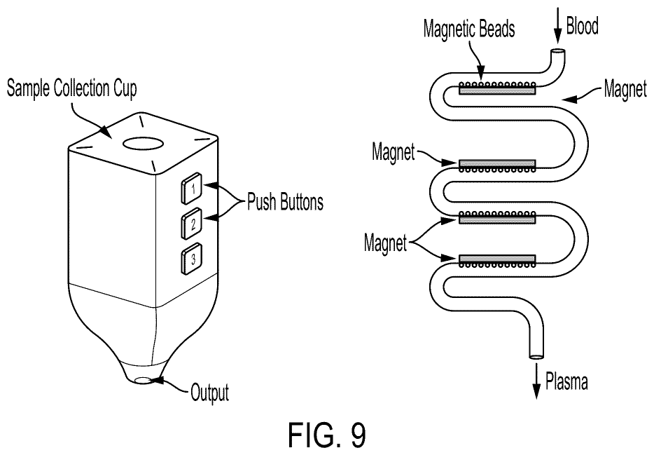

A device can comprise, for example, a serpentine channel tube, housed in a unit that has push buttons, each push button configured to release a corresponding reagent (e.g., aggregate agent, antibodies) (FIG. 9).

The serpentine channel tube can have two openings, wherein one opening is to input fluid sample, magnetic beads, and aggregation agent, and the other opening is to collect the supernatant. In some embodiments, the channel is lined with magnets, wherein the magnets can capture magnetic beads with target biological entities.

### Device Using Fingerstick

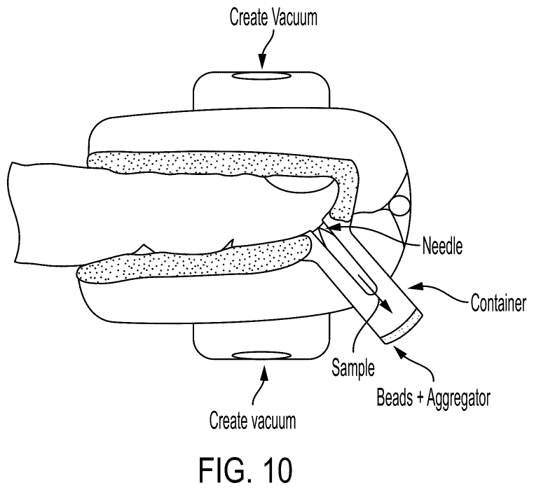

A device can comprise, for example, a finger sleeve with an opening for needle prick and collection of blood sample (FIG. 10).

The finger sleeve can have two buttons on the side that can be pressed simultaneously to create a vacuum and drive the needle into the finger. In some embodiments, the finger sleeve is connected to a container that can collect blood sample. In some embodiments, the container can have magnetic beads and/or aggregate agent to mix with the collected blood sample.

### Device Using a Dropper Tube

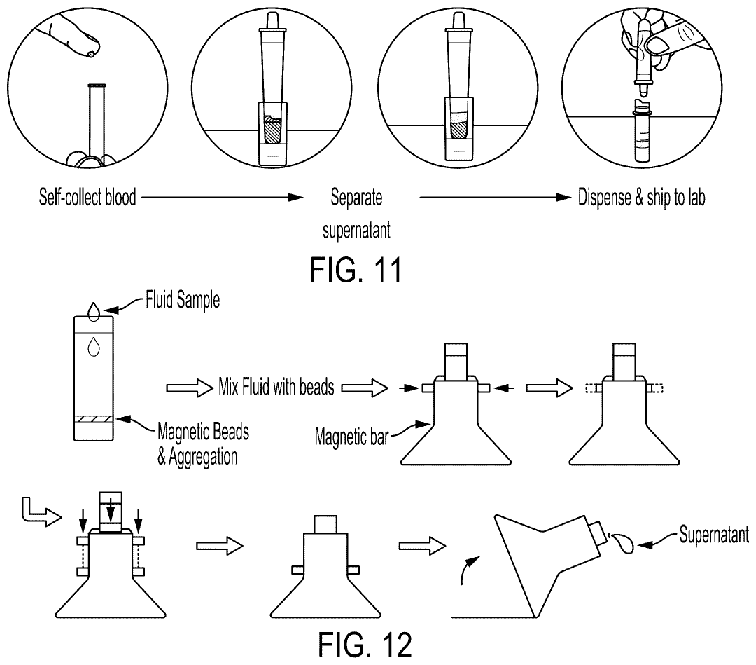

A device can comprise, for example: (a) a tube; (b) a lid to contain a fluid sample; and (c) a source of a magnetic field in proximity to the tube (FIG. 11).

In some embodiments, the tube can be at least about 20 mm, at least about 30 mm, at least about 40 mm, at least about 45 mm, or at least about 50 mm in length. The diameter can be at least about 13 mm, at least about 14 mm, at least about 15 mm, at least about 16 mm, or at least about 17 mm in diameter. In some embodiments the bottom diameter of the tube is smaller than the top diameter of the tube. In some embodiments, the top diameter of the tube is smaller than the bottom diameter of the tube.

The tube can hold the sample fluid, magnetic beads, and aggregation agent. In some embodiments, the tube is a packed tube comprising the magnetic beads and aggregation agent. In some embodiments, the magnetic beads and aggregation agent are lyophilized. In some embodiments, the tube is a packed tube comprising lyophilized magnetic beads and aggregation agent. In some embodiments, the tube can have one opening at the top of the tube to decant the supernatant. In some embodiments, the tube can be made from a flexible or deformable material (e.g., plastic), wherein the tube can be squeezed to dispense supernatant from the top opening of the tube. In some embodiments, the tube can have an inner wall that is functionalized with a hydrophobic coating (e.g., silica, Teflon, fluoropolymers). In some embodiments, the tube can have an inner wall that is functionalized to be non-wettable. In some embodiments, the tube can have an inner wall that is coated with a chelation agent.

In some embodiments, the tube lid can include a size-exclusion membrane positioned near the outlet of the lid to capture residual cells in the supernatant. In some embodiments, the lid further comprises a size-exclusion membrane in fluid communication with the dispensing tip. In some embodiments, the tube lid can comprise a filter. In some embodiments, the lid further comprises a filter in fluid communication with the dispensing tip. In some embodiments, the filter can be hydrophilic or hydrophobic. In some embodiments, the filter can be coated with magnetic beads, aggregation agents, or any combination of them. In some embodiments, the filter is one layer or a plurality of different layers.

In some embodiments, the tube lid can comprise a dispensing tip. In some embodiments, the dispensing tip only dispenses a pre-determined amount of supernatant.

In some embodiments, the device includes a storage vessel. In some embodiments, the storage vessel consists of a porous wicking membrane. In some embodiments, the porous wicking membrane is only capable of accepting a fixed amount of supernatant.

In some embodiments, the magnetic field can be generated by a single rare earth magnet or a plurality of magnets or electromagnet or any variations thereof. The single rare earth magnet or electromagnet can be in the shape of a tube holder to hold the tube in place and attract magnetic beads. In some embodiments, the magnet is disposed in a bracket, wherein the bracket comprises a receptacle suitable for holding the packed tube in proximity to the magnet. In some embodiments, the magnetic field can be a neodymium magnet. In some embodiments, the source of the magnetic field includes a visual indicator that assists the user in determining the end point of the capture and separation process. In some embodiments, the visual indicator consists of a small light source programmed to change states at the end of the process. In some embodiments, the light source is automatically programmed to activate once the tube is placed correctly in the magnetic stand.

### Device With Pinch Mixing

A device can comprise, for example: (a) a tube; (b) a tube lid with a dispensing tip; (c) a tube holder with buttons that can be depressed to move in the radial direction and translated along axial direction and (d) a source of a magnetic field at the bottom of the magnetic holder (FIG. 12).

In some embodiments, the tube is a packed tube comprising the magnetic beads and aggregation agent. In some embodiments, the magnetic beads and aggregation agent are lyophilized. In some embodiments, the tube is a packed tube comprising lyophilized magnetic beads and aggregation agent. The tube holder can have buttons that can be depressed to induce mixing of the sample fluid, magnetic beads, and aggregation agent. In some embodiments the buttons are configured to move in the axial plane and induce a relative motion between the tube and the magnetic source.

In some embodiments, the magnetic field can be generated by a single rare earth magnet or a plurality of magnets or electromagnet or any variations thereof. The single rare earth magnet or electromagnet can be in the shape of a vessel holder to hold the tube in place and attract magnetic beads. In some embodiments, the magnet is disposed in a bracket, wherein the bracket comprises a receptacle suitable for holding the packed tube in proximity to the magnet. In some embodiments, the magnetic field can be a neodymium magnet.

In some embodiments, the tube holder includes a visual indicator that assists the user in determining the end point of the capture and separation process. In some embodiments, the visual indicator may consist of a small light source programmed to change states at the end of the process. In some embodiments, the light source is automatically programmed to activate once the tube is placed correctly in the magnetic stand.

### Device Using Pierce Feature and a Transfer Zone

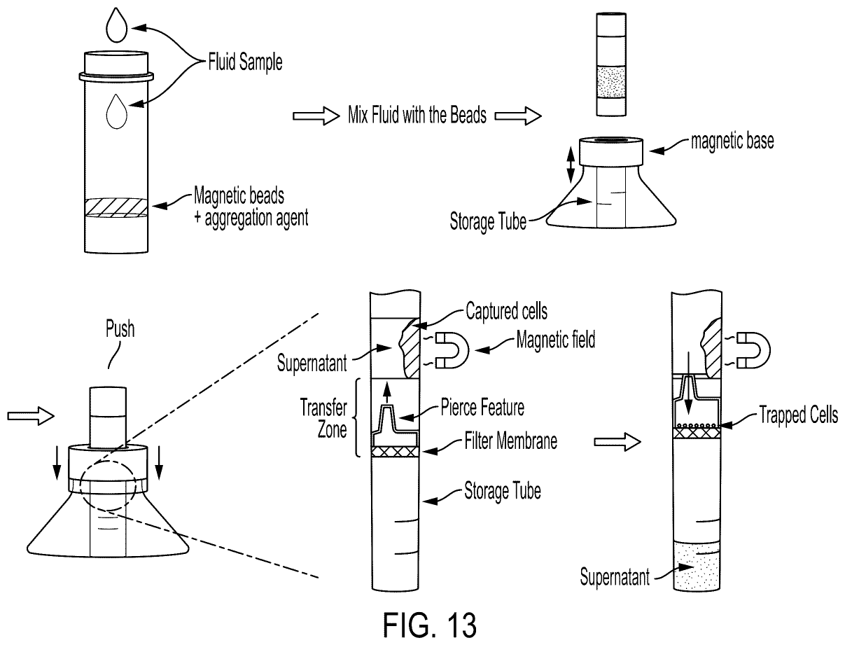

A device can comprise, for example, (a) a collection tube to receive the sample; (b) a tube holder; and (c) a storage tube (FIG. 13). The tube can hold the sample fluid, magnetic beads, and aggregation agent. In some embodiments, the tube is a packed tube comprising the magnetic beads and aggregation agent. In some embodiments, the magnetic beads and aggregation agent are lyophilized. In some embodiments, the tube is a packed tube comprising lyophilized magnetic beads and aggregation agent. The tube holder is configured in a manner to receive the collection tube that orients the body of the device in proximity to a pierce feature that can create an opening in the body of the collection tube. The opening can be created in such a manner that the supernatant in the tube is drawn into a transfer zone wherein the supernatant is subsequently transferred into the storage tube. In some embodiments, the transfer occurs due to gravity alone. In some embodiments, the transfer is initiated or expedited with a positive pressure. In some embodiments, a filter membrane can be incorporated in the transfer zone to trap additional cellular material.

### Magnetic Bead-Based Capture System

Disclosed herein is a system relating to the magnetic bead-based capture of a biological entity. This system includes at least one device according to the present disclosure and a plurality of magnetic beads, each functionalized to bind to the target biological entity to separate the biological entity from the fluid sample.

In some embodiments, the fluid sample can be venous blood, capillary blood, or another bodily fluid.

In some embodiments, the fluid sample can be mixed with an anticoagulant (e.g., EDTA, sodium citrate, heparin, etc.) to inactivate clotting. In some embodiments, the fluid sample can be mixed with a calcium chelation agent or chelator (e.g., EDTA, EGTA, sodium citrate, trisodium citrate, oxalates, BAPTA, BAPTA tetrapotassium salt, BAPTA tetracesium salt, BAPTA tetrasodium salt, BAPTA AM, 5,5′-Dimethyl BAPTA, AM, 5,5′-Difluoro BAPTA, or 5,5′-Dibromo BAPTA) is used to chelate calcium to prevent the activation of serum complement. In some embodiments, the chelation enhances the efficiency of the magnetic bead-based capture. In some embodiments, the anticoagulant and the calcium chelation agent or chelator comprise the same material. In some embodiments, the anticoagulant and the calcium chelation agent or chelator are different materials or a combination thereof and the calcium chelation agent or chelator is only added if the anticoagulant does not inherently possess chelation properties (e.g., thrombin-inactivation anticoagulants).

In some embodiments, the fluid sample can be mixed with a protective agent to preserve and stabilize cells (e.g., blood cells). In some embodiments, the protective agent can be a preserving agent and an anticoagulant. In some embodiments, the preserving agent is aldehyde, oxazolidine, alcohol, cyclic urea, or any combination thereof. In some embodiments, the preserving agent can be imidazolidinyl urea or diazolidinyl urea. In some embodiments, the anticoagulant is K3EDTA.

In some embodiments, the biological entity can be red blood cells, white blood cells, platelets, and/or any combination thereof.

In some embodiments, the magnetic beads can be functionalized with an antibody and/or capture agents. In some embodiments, the magnetic beads are functionalized to capture albumin, globulin, fibrinogen, hemoglobin, or any combination thereof.

In some embodiments, the antibody and/or capture agents functionalized to the magnetic beads can simultaneously bind to multiple biological entities (e.g., red blood cells, white blood cells and platelets) or can bind the cells individually.

In some embodiments, the system can include an aggregation agent used to aggregate the target biological entities prior to or in parallel to the capture and separation of the target biological entities from the fluid sample.

In some embodiments, the fluid sample can be combined with a polyionic polymer as an aggregation agent. The fluid sample containing the particles and the polyionic polymer can be allowed to incubate for a time sufficient for aggregation of the particles to occur. A reversing agent capable of cleaving the polyionic polymer can be used to reverse the aggregation. In some embodiments, the reversing agent can be a chemical compound, composition, or material, either naturally occurring or synthetic, organic, or inorganic, capable of reversing the aggregation of particles by at least partial depolymerization of the polyionic polymer.

In some embodiments, the reversing agent can be a buffer or chemical that breaks protein-protein bonds. In some embodiments, that buffer or chemical can be an IgG elution buffer, 100 mM glycine-HCl (pH 2.5-3.0), 100 mM citric acid (pH 3.0), 50-100 mM triethylamine or triethanolamine (pH 11.5), 150 mM ammonium hydroxide (pH 10.5), 0.1 M glycine-NaOH (pH 10.0), gentle Ag/Ab elution buffer, 5 M lithium chloride, 3.5 M magnesium or potassium chloride, 3.0 M potassium chloride, 2.5 M sodium or potassium iodide, 0.2-3.0 M sodium thiocyanate, 0.1 M Tris-acetate with 2.0 M NaCl (pH 7.7), 2-6 M guanidine-HCl, 2-8 M urea, 1.0 M ammonium thiocyanate, 1% sodium deoxycholate, 1% SDS, 10% dioxane, 50% ethylene glycol (pH 8-11.5), or >0.1 M counter ligand or analog.

In some embodiments, the aggregation agent can be an antibody (e.g., polyclonal, or monoclonal antibody) or protein that can bind to a target biological entity. For example, an antibody can bind to CD44, CD45, CD46, CD47, CD29, CD35, and/or CD82, wherein the target entity is a white blood cell, and other antibodies can be selected to bind other known antigens expressed on the biological entity (e.g., on the cell surface). In some embodiments, the aggregation agent comprises a monoclonal, a polyclonal antibody, or combination thereof that can bind to CD44, CD45, CD46, CD47, CD29, CD35, CD82 and/or CD235a. In some embodiments, the aggregation agent can bind to multiple cell types simultaneously. In some embodiments, the aggregation agent is functionalized on the surface of the magnetic bead.

In some embodiments, the aggregation agent can be an antibody that binds to proteins non-specifically. For example, an antibody can bind to albumin, globulin, hemoglobin and fibrinogen, wherein the sample fluid is venous and/or capillary blood or other biological fluid with these proteins.

In some embodiments, the aggregation agent can be an antibody that binds to the proteins or cellular material in the fibrin clot or platelets, red blood cells, or white blood cells in fibrin clot or any combination thereof. In some embodiments, the aggregation agent can be an antibody that binds the exterior of the clot or the surface of red blood cells, white blood cells, platelets, or any combination thereof. In some embodiments, the magnetic capture and separation of biological entity (e.g., red blood cells, white blood cells, and/or platelets) from fluid sample (e.g., blood) can prevent the formation of fibrin clots without addition of other anticoagulants. In some embodiments, the magnetic capture and separation of the biological entity occurs after the formation of a full fibrin clot.

In some embodiments, the aggregation agent can be a clot-activator, human fibrin, and/or an anti-fibrin antibody that can expedite the formation of fibrin clot within the fluid sample.

In some embodiments, the aggregation agent can be an anti-hemoglobin antibody.

In some embodiments, the aggregation agent can be a platelet aggregation inducer (e.g., adenosine diphosphate, collagen, arachidonic acid, thrombin, epinephrine, and/or ristocetin).

In some embodiment, the concentration of aggregation agent used can be at least about 1 mg/mL, at least about 2 mg/mL, at least about 3 mg/mL, at least about 4 mg/mL, at least about 5 mg/mL, at least about 6 mg/mL, at least about 7 mg/mL, at least about 8 mg/mL, at least about 9 mg/mL, or at least about 10 mg/mL. The concentration of aggregation agent used can be about 1 mg/mL to about 6 mg/mL. In some embodiments, the concentration of aggregation agent used can be about 4 mg/mL to 6 mg/mL. In some embodiments, the concentration of aggregation agent used can be about 3 mg/mL to 5 mg/mL. In some embodiments, a solvent used to prepare the aggregation agent can be water or water with buffering salts.

In some embodiments, the plurality of magnetic beads and the aggregation agent can be provided separately or together in a storage buffer.

In some embodiments, the plurality of magnetic beads and the aggregation agent can be mixed prior to adding the fluid sample.

In some embodiments, the plurality of magnetic beads and the aggregation agent can be dried directly in a collection tube or vessel prior to adding the fluid sample.

In some embodiments, the plurality of magnetic beads and the aggregation agent can be lyophilized directly in a tube or vessel prior to adding the fluid sample.

In some embodiments, the plurality of magnetic beads and the aggregation agent can be added to the fluid sample separately, one after the other.

In some embodiments, the plurality of magnetic beads and the aggregation agent can be contained in the tube or vessel of the device disclosed herein (e.g., packed tube), wherein the tube or vessel also holds the fluid sample during the operation of the device.

In some embodiments, the plurality of magnetic beads can be bound to the surface of a functionalized non-magnetic microbead of larger diameter (e.g., 1-fold, 2-fold, 3-fold, 4-fold, 5-fold, 6-fold, 8-fold, 9-fold, or 10-fold larger than that of the magnetic beads) to facilitate the capture of biological entities from fluid samples.

In some embodiments, the magnetic beads can be no larger than 0.25 μm, 0.55 μm, 1 μm, 2 μm, 3 μm, or 4 μm. In some embodiments, the plurality of magnetic beads has a mean greatest diameter of no larger than 4 μm.

In some embodiments, the concentration of the magnetic beads used can be at least about 50 mg/mL, at least about 100 mg/mL, at least about 150 mg/mL, at least about 200 mg/mL, at least about 250 mg/mL or at least about 300 mg/mL.

In some embodiments, the plurality of magnetic beads can be ferromagnetic, paramagnetic, or superparamagnetic.

In some embodiments, the plurality of magnetic beads can be passively absorbed onto the surface of the larger non-magnetic microbead.

In some embodiments, the larger microparticle can be magnetic.

In some embodiments, the captured biological entities can be released from the plurality of magnetic beads for further analysis.

In some embodiments, the compartment disclosed herein can be a tube or vessel or other container that can hold the fluid sample, magnetic beads, and aggregation agent. In some embodiments, the tube or vessel or other container can be a packed tube, packed vessel or other packed container comprising the plurality of magnetic beads and the aggregation agent. In some embodiments, the magnetic beads and aggregation agent are lyophilized. In some embodiments, the packed tube comprising lyophilized magnetic beads and aggregation agent. In some embodiments, the tube can be coated in the interior surface with an antibody and/or capture agent that can bind to the surface of the target entity. In some embodiments, the antibody and/or capture agent can be at the bottom half of the tube. In some embodiments, the tube of the device is coated with a hydrophobic material. In some embodiments, the tube is made of a hydrophobic material. In some embodiments, the tube is made of a deformable material (e.g., plastic). In some embodiments, the tube of the device is coated with an anticoagulant. In some embodiments, the tube of the device contains a chelation agent. In some embodiments, the chelation agent chelates calcium to enhance the function of the aggregation agent.

In some embodiments, the system disclosed herein can comprise a source of a magnetic field. The magnetic field can be introduced to the fluid sample to separate magnetic bead-captured material from non-captured material. The magnetic field can be generated by a single rare earth magnet or electromagnet or any variation thereof. In some embodiments, the magnetic field can be a neodymium magnet.

### Magnetic Bead-Based Capture Methods

Disclosed herein are methods relating to the magnetic bead-based capture of biological entity. Disclosed herein is a method of using a device to capture target a biological entity from a fluid sample. In some embodiments, the method disclosed herein can be used to capture a target biological entity from a fluid sample.

### Device Using Piezoelectric Buzzer

An example method of using the device comprises the steps of: (a) adding the fluid sample into a tube containing a plurality of magnetic beads functionalized to bind to the target biological entity to the tube and/or aggregation agent (e.g., a packed tube); (b) operating the device by turning on and off the piezoelectric buzzer to mix the fluid sample and magnetic beads; and (c) collecting the separated supernatant and/or magnetic bead-captured target biological entities. In some embodiments, the piezoelectric buzzer can be turned on until the thixotropic gel is liquified and mixed with the fluid sample, magnetic beads, and/or aggregation agent. In some embodiments, the piezoelectric buzzer can be turned off after mixing sample fluid and magnetic beads to allow that the gel migrates and solidifies between the target biological entities and supernatant.

### Device Using Fluidic Serpentine Channel

An example method of using the device comprises the steps of: (a) adding the fluid sample with a plurality of magnetic beads functionalized to bind the target biological entity and/or aggregation agent to the fluidic serpentine channel; (b) applying a magnetic field toward the end of the channel to capture target biological entity; and (c) collecting the separated supernatant and/or magnetic beads-capture target biological entity from the output.

### Device Using Plastic Disposable Housing Unit

An example method of using the device comprises the steps of: (a) adding the fluid sample into a tube containing a plurality of magnetic beads functionalized to bind the target biological entity and/or aggregation agent to the tube (e.g., a packed tube); (b) introducing magnetic field to the bottom of the tube; (c) inverting the tube into the housing unit, with magnetic field still in proximity to the bottom of the tube to separate biological entities from the fluid sample; (d) removing the tube after separation; and (e) putting the cap on the housing unit to prevent air from entering.

### Device Using Tube That Directly Collects Fluid Sample

An example method of using the device comprises the steps of: (a) collecting fluid sample from the capillary tube inserted in the lid of a tube that contains a plurality of magnetic beads and/or aggregation agent (e.g., a packed tube); (b) mixing fluid sample, magnetic beads, and/or aggregation agent in the tube; (c) introducing a magnetic field to the tube; and (d) opening the lid to decant the supernatant in another tube. In some embodiments, the supernatant can be decanted with or without applying a magnetic field. In some embodiments, instead of opening the lid at the top to decant the supernatant, the tube can have an opening at the bottom of the tube, wherein the tube can be flexible and can be squeezed to dispense the supernatant.

In some embodiments, mixing or agitating the fluid sample, magnetic beads, and/or aggregation agent in the tube can be performed by shaking the tube, inverting the tube, rolling the tube, rotating the tube, tapping the exterior of the tube, or any combination thereof.

In some embodiments, the magnetic field can be introduced in the form of a tube holder that can be made from a single rare earth magnet, electromagnet, or variations thereof, wherein the tube can sit inside the tube holder. In some embodiments, the magnetic field can be a neodymium magnet.

### Device Using High Surface Magnetic Base

An example method of using the device comprises the steps of: (a) adding the fluid sample into a tube or vessel containing with a plurality of magnetic beads functionalized to bind the target biological entity and/or aggregation agent (e.g., a packed tube or packed vessel), with magnet at the base of the tube or vessel; (b) mixing the fluid sample, magnetic beads, and/or aggregation agent in the tube; (c) inverting the tube or vessel; and (d) applying pressure to the tube or vessel to dispense supernatant from the outlet.

### Device Using Two Compartments

An example method of using the device comprises the steps of: (a) adding the fluid sample into a large compartment containing a plurality of magnetic beads functionalized to bind the target biological entity and/or aggregation agent; (b) mixing fluid sample, magnetic beads, and/or aggregation agent in the tube; (c) applying a magnetic field to the smaller compartment to collect biological entities captured by magnetic beads; (d) pushing by physical actuation a physical pin to the middle section of connecting tube, placed between the two compartments, to create a seal; and (e) dispensing the supernatant from the large compartment. In some embodiment, the physical pin can be magnetic, wherein the method comprises introducing a magnetic field to the connecting tube to pinch the connecting tube tight.

### Device Using Toggling Between Two States

An example method of using the device comprises the steps of: (a) adding the fluid sample into a tube containing a plurality of magnetic beads functionalized to bind the target biological entity and/or aggregation agent (e.g., a packed tube); (b) mixing fluid sample, magnetic beads, and/or aggregation agent in the tube; (c) placing the tube in a housing unit with two buttons on the sides and a magnet at the bottom; (d) simultaneously depressing the two buttons to bring a magnet in contact with the bottom of the tube; and (e) dispensing the supernatant from the top opening of the tube by inversion. In some embodiment, the housing unit can have a threaded screw assembly, wherein the method comprises turning or twisting a screw into the screw assembly to bring a magnet in contact with the bottom of the tube. In some embodiments, the screw can be turned one quarter of one full rotation, one half of a full rotation, three quarters of a full rotation or one full rotation to bring the magnet into contact with the bottom of the tube.

### Device Using Spring Loaded Cap

An example method of using the device comprises the steps of: (a) adding the fluid sample into a tube containing with a plurality of magnetic beads functionalized to bind the target biological entity and/or aggregation agent, wherein the tube further comprises a magnet at the bottom; (b) mixing fluid sample, magnetic beads, and/or aggregation agent in the tube; (c) pushing down a flexible, air-filled bulb and rigid hollow rod into the spring-loaded cap of the tube; (d) releasing the bulb to collect the supernatant up the hollow rod; and (e) dispensing the supernatant to another container.

### Device With Push Buttons

An example method of using the device comprises the steps of: (a) adding the fluid sample with a plurality of magnetic beads functionalized to bind the target biological entity and/or aggregation agent to the device; (b) mixing fluid sample, magnetic beads, and/or aggregation agent in the tube; (c) depressing buttons that release reagents and create positive pressure for fluid to be pushed through a channel; and (d) collecting the supernatant dispensed from the device.

### Device Using Fingerstick

An example method of using the device comprises the steps of: (a) inserting a finger into the device, with collection tube attached, (b) pressing two buttons simultaneously on the side of the device to create vacuum and drive a needle into the finger; (c) unscrewing the tube with blood, magnetic beads, and/or aggregate agent from the device; and (d) placing the tube into a magnetic stand to separate target biological entity from the blood sample.

### Device Using a Dropper Tube

An example method of using the device comprises the steps of: (a) disposing the fluid sample to a packed tube or packed vessel packed with a plurality of magnetic beads functionalized to bind the target biological entity and/or an aggregation agent; (b) after the disposing the fluid sample into the packed tube, capping the packed tube with a lid, wherein the lid comprises a dispensing tip; (c) after the capping the packed tube with the lid, agitating the packed tube to mix the fluid sample with the plurality of magnetic beads, and/or aggregation agent; (d) after the agitating the packed tube, disposing the packed tube or packed vessel in proximity of a magnetic field (e.g., magnetic holder, magnetic sleeve) wherein the magnetic field separates the magnetic beads form a supernatant; (e) inverting or tilting the tube or vessel to separate the captured cells from the supernatant; and (f) applying pressure (e.g., squeezing) to the external wall of the tube or vessel to dispense the supernatant from the dispensing tip into a collection tube. The supernatant can be dispensed through the dispensing tip into a collection tube.

In some embodiments, a package contains the packed tube. In some embodiments, the method comprises removing the packed tube from the package before disposing the fluid sample into the packed tube. In some embodiments, the package can be made of an aluminum bag, plastic bag, box (e.g., cardboard box), paper or a combination thereof. In some embodiments, the package can comprise packaging foams (e.g., expanded polystyrene, PE, PU, cross-linked) to protect items (e.g., packed tube) within the package. In some embodiments, the package can be made from recyclable materials. In some embodiments, the package can be recycled. In some embodiments, the package can be stackable. In some embodiment, the package can be compartmentalized to fit items (e.g., packed tube) of various shapes (e.g., round, oblong, flat, etc.). In some embodiments, the packaging can comprise a desiccant.

In some embodiments, the method comprises lyophilizing the plurality of magnetic beads and the aggregation agent prior to adding the fluid sample into the packed tube. In some embodiments, the magnetic beads and aggregation agent are lyophilized. In some embodiments, the packed tube comprises lyophilized magnetic beads and aggregation agent.

In some embodiments the supernatant is dispensed into a vessel used to store the supernatant. In some embodiments, the supernatant is dispensed into a sample input zone to start a diagnostic test. In some embodiments, the supernatant is dispensed onto a wicking membrane.

In some embodiments, agitating the packed tube can be achieved by shaking, rolling, or inverting the tube or a combination of movements generated by a force provider. In some embodiments, agitating is used to homogenize the magnetic bead, the aggregation agent, and the fluid sample. In some embodiments, agitating is used to cause the aggregation of the captured biological entities with the aggregation agent. In some embodiments, agitating is used to homogenize the magnetic bead, aggregation agent, and fluid sample to cause the aggregation of the biological entities.

In some embodiments, the lid can include a size-exclusion membrane positioned near the outlet of the lid to capture residual cells in the supernatant. In some embodiments, the lid further comprises a size-exclusion membrane in fluid communication with the dispensing tip. In some embodiments, the tube lid can comprise a filter. In some embodiments, the lid further comprises a filter in fluid communication with the dispensing tip. In some embodiments, the filter can be hydrophilic or hydrophobic. In some embodiments, the filter can be coated with magnetic beads, aggregation agents, or any combination of them. In some embodiments, the filter is one layer or a plurality of different layers.

In some embodiments, the lid can comprise a dispensing tip. In some embodiments, the dispensing tip only dispenses a pre-determined amount of supernatant.

In some embodiments, the plurality of magnetic beads is substantially separated from the supernatant within one minute of disposing the packed tube in proximity of the magnetic field. In some embodiments, the plurality of magnetic beads is substantially separated from the supernatant within at least about 5 seconds, at least about 10 seconds, at least about 15 seconds, at least about 20 seconds, at least about 25 seconds, at least about 30 seconds, at least about 35 seconds, at least about 40 seconds, at least about 45 seconds, at least about 50 seconds, at least about 55 seconds, at least about 1 minute, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, or at least about 1 hour of disposing the packed tube in proximity of the magnetic field.

In some embodiments, at least about 80%, at least about 85%, at least about 90%, at least about 95%, at least about 99.9% at least 100% of the target biological entity is separated from the supernatant. In some embodiments, at least about 95% of the target biological entity is separated from the supernatant. In some embodiments, at least about 99% of the target biological entity is separated from the supernatant. In some embodiment, the target biological entity is captured with at least about 80%, at least about 85%, at least about 90%, at least about 95%, at least about 99.9% at least 100% efficiency. In some embodiments, the target biology entity is captured with 80%, at least about 85%, at least about 90%, at least about 95%, at least about 99.9% at least 100% purity.

### Device With Pinch Mixing

An example method of using the device comprises the steps of: (a) adding the fluid sample to a packed tube or packed vessel with a mixture comprising: a plurality of magnetic beads functionalized to bind the target biological entity and/or aggregation agent (b) placing the tube in a tube holder with compressible buttons that move up and down; (c) mixing the fluid sample, magnetic beads, and/or aggregation agent in the tube by pushing on the compressible buttons; (d) pushing the sides of the tube with the compressible buttons and sliding it down the tube holder (e.g., side slits) to come in contact with a source of a magnetic field at the bottom of the tube holder bottom to separate the fluid sample (e.g., plasma); (e) inverting the tube or vessel; and (f) applying pressure to the tube or vessel to dispense supernatant from the outlet.

### Device Using Pierce Feature and a Transfer Zone

An example method of using the device comprises the steps of (a) adding the fluid sample to a packed tube with a mixture, comprising a plurality of magnetic beads functionalized to bind the target biological entity and/or aggregation agent; (b) mixing the fluid sample with the magnetic beads and aggregation agent; (c) placing the tube in a tube holder with a pierce feature and a transfer zone to initiate the separation of a supernatant; (d) using the pierce feature in the tube holder to create an opening in the collection tube to initiate the transfer of the supernatant; and (e) collecting the supernatant in the storage tube. In some embodiments, the pierce feature is initiated by inducing relative motion between the piercing feature and the storage tube. In some embodiments, the opening is created on the bottom portion of the body of the tube. In some embodiments, the supernatant collects in the transfer zone and subsequently in the storage tube by gravity alone. In some embodiments, a positive pressure is applied to the collection tube to the collection tube to initiate or expedite the transfer of the supernatant.

The methods of using the disclosed devices can further comprise collecting the supernatant in a separate tube or vessel and analyzing the supernatant and/or the capture target biological entity using one or more diagnostic tools or technique (e.g., qPCR, western blot, PCR, immunoassay, or sequencing). In some embodiments, the captured biological entities can be released from the plurality of magnetic beads for further analysis.

In some embodiments, the fluid sample used in any of the methods disclosed herein can be blood (e.g., venous blood, capillary blood) or another bodily fluid.

In some embodiments, the fluid sample used in any of the methods disclosed herein can be mixed with an anticoagulant (e.g., EDTA, sodium citrate, heparin, etc.) to inactivate clotting prior to or in parallel to capture and separation. In some embodiments, the fluid sample can be mixed with a calcium chelation agent or chelator (e.g., EDTA, EGTA, sodium citrate, trisodium citrate, oxalates, BAPTA, BAPTA tetrapotassium salt, BAPTA tetracesium salt, BAPTA tetrasodium salt, BAPTA AM, 5,5′-Dimethyl BAPTA, AM, 5,5′-Difluoro BAPTA, or 5,5′-Dibromo BAPTA) to chelate calcium to prevent the activation of serum complement. In some embodiments, the anticoagulant and the calcium chelation agent or chelator comprise the same material. In some embodiments the calcium chelation agent enhances the function of capture and separation process. In some embodiments, the anticoagulant and the calcium chelation agent or chelator are different materials or a combination of them and the calcium chelation agent or chelator is only added if the anticoagulant does not inherently possess chelation properties (e.g., thrombin-inactivation anticoagulants).

In some embodiments, the fluid sample used in any of the methods disclosed herein can be mixed with a protective agent to preserve and stabilize cells (e.g., blood cells). In some embodiments, the protective agent can be a preserving agent and an anticoagulant. In some embodiments, the preserving agent is aldehyde, oxazolidine, alcohol, cyclic urea, or any combination thereof. In some embodiments, the preserving agent can be imidazolidinyl urea or diazolidinyl urea. In some embodiments, the anticoagulant is K3EDTA.

In some embodiments, the biological entity can be red blood cells, white blood cells, platelets, and/or any combination thereof.

In some embodiments, the magnetic beads used in any of the methods disclosed herein can be functionalized with an antibody and/or capture agents. In some embodiments, the magnetic beads are functionalized to capture albumin, globulin, fibrinogen or hemoglobin or any combination thereof.

In some embodiments, the antibody and/or capture agents functionalized to the magnetic beads can simultaneously bind to red blood cells, white blood cells, and platelets or can bind the cells individually or can bind any combination thereof.

In some embodiments, the method can include aggregating the target biological entities with the aggregation agent prior to the capture and separation of the target biological entity from the fluid sample. In some embodiments, the fluid sample can be combined with a polyionic polymer as an aggregation agent. The fluid sample containing the particles and the polyionic polymer can be allowed to incubate for a time sufficient for aggregation of the particles to occur. A reversing agent capable of cleaving the polyionic polymer can be used to reverse the aggregation. In some embodiments, the reversing agent can be a chemical compound, composition, or material, either naturally occurring or synthetic, organic, or inorganic, capable of reversing the aggregation of particles by at least partial depolymerization of the polyionic polymer.

In some embodiments, the reversing agent can be a buffer or chemical that breaks protein-protein bonds. In some embodiments, that buffer or chemical can be an IgG elution buffer, 100 mM glycine-HCl (pH 2.5-3.0), 100 mM citric acid (pH 3.0), 50-100 mM triethylamine or triethanolamine (pH 11.5), 150 mM ammonium hydroxide (pH 10.5), 0.1 M glycine-NaOH (pH 10.0), gentle Ag/Ab elution buffer, 5 M lithium chloride, 3.5 M magnesium or potassium chloride, 3.0 M potassium chloride, 2.5 M sodium or potassium iodide, 0.2-3.0 M sodium thiocyanate, 0.1 M Tris-acetate with 2.0 M NaCl (pH 7.7), 2-6 M guanidine-HCl, 2-8 M urea, 1.0 M ammonium thiocyanate, 1% sodium deoxycholate, 1% SDS, 10% dioxane, 50% ethylene glycol (pH 8-11.5), or >0.1 M counter ligand or analog. In some embodiments, the aggregation agent can be an antibody (e.g., polyclonal or monoclonal antibody) or protein that can bind to a target biological entity. For example, an antibody can bind to CD44, CD45, CD46, CD47, CD29, CD35, CD82, CD50, CD132, CD156c, CD162, CD256, CD262, CD289, CD321, CD329, CD355, and/or CD357, wherein the target entity is a white blood cell. In another example, an antibody can bind to CD 35, CD 47, CD75, CD108, CD 139, CD 174, CD233, CD234, CD235a, CD235b, CD236, CD238, CD239, CD240CE, CD240D, CD241, CD242 or CD 297, wherein the target entity is a red blood cell. In another example, an antibody can bind to CD9, CD17, CD23, CD31, CD32, CD42a, CD42b, CD42c, CD42d, CD47, CD49e, CD49f, CD60a, CD60b, CD60c, CD61, CD62P, CD63, CD66e, CD82, CD84, CD102, CD107a, CD107b, CD109, CD110, CD150a, CD140b, CD141, CD154, CD165, CD173, CD194, CD324, wherein the target entity is an activated or inactivated platelet. Other antibodies can be selected to bind other known antigens expressed on the biological entity (e.g., on the cell surface). In some embodiments, the antibody can bind to multiple cell types simultaneously. In some embodiments, the antibody can be functionalized on the surface of the magnetic bead. In some embodiments, the aggregation agent can be an antibody that binds to proteins non-specifically. For example, an antibody can bind to albumin, globulin, hemoglobin and fibrinogen, wherein the target entity is venous and/or capillary blood or other biological fluid with these proteins. In some embodiments, the aggregation agent can be an antibody that binds to the proteins or cellular material in the fibrin clot or platelets, red blood cells, or white blood cells in fibrin clot or any combination thereof. In some embodiments, the aggregation agent can be an antibody that binds the exterior of the clot or the surface of red blood cells, white blood cells, platelets, or any combination thereof. In some embodiments, the magnetic capture and separation of the biological entity (e.g., red blood cells, white blood cells, and/or platelets) from the fluid sample (e.g., blood) can prevent the formation of fibrin clot without addition of other anticoagulants. In some embodiments, the magnetic capture and separation of the biological entity can occur after the formation of a full fibrin clot. In some embodiments, the aggregation agent can be a clot-activator, human fibrin, and/or an anti-fibrin antibody that can expedite the formation of fibrin clot within the fluid sample. In some embodiments, the aggregation agent can be a platelet aggregation inducer (e.g., adenosine diphosphate, collagen, arachidonic acid, thrombin, epinephrine, and/or ristocetin). In some embodiment, the concentration of aggregation agent used can be at least about 1 mg/mL, at least about 2 mg/mL, at least about 3 mg/mL, at least about 4 mg/mL, at least about 5 mg/mL, at least about 6 mg/mL, at least about 7 mg/mL, at least about 8 mg/mL, at least about 9 mg/mL, or at least about 10 mg/mL. In some embodiment, the concentration of aggregation agent used can be about 1 mg/mL to about 6 mg/mL. In some embodiments, the concentration of aggregation agent used can be about 4 mg/mL to 6 mg/mL. In some embodiments, the concentration of aggregation agent used can be about 3 mg/mL to 5 mg/mL. In some embodiments, a solvent used to prepare the aggregation agent can be water or water with buffering salts.

In some embodiments, the plurality of magnetic beads and the aggregation agent can be provided separately or together in a storage buffer.

In some embodiments, the plurality of magnetic beads and the aggregation agent can be mixed prior to adding the fluid sample.

In some embodiments, the plurality of magnetic beads and the aggregation agent can be dried directly in a collection tube or vessel prior to adding the fluid sample.

In some embodiments, the plurality of magnetic beads and the aggregation agent can be added to the fluid sample separately, one after the other.

In some embodiments, the plurality of magnetic beads and the aggregation agent can be contained in the tube or vessel of the device disclosed herein, wherein the tube or vessel also holds the fluid sample during the operation of the device.

In some embodiments, the magnetic field used in any of the methods disclosed herein can be introduced in the form of a tube holder that can be made from a single rare earth magnet, electromagnet, or variations thereof, wherein the tube can sit inside the tube holder. In some embodiments, the magnetic field is a magnet. In some embodiments, the magnet is disposed in a bracket, wherein the bracket comprises a receptacle suitable for holding the packed tube in proximity to the magnet. In some embodiments, the magnetic field can be a neodymium magnet.

### Kit

Disclosed herein is a kit relating to the magnetic bead-based capture of a biological entity. In some embodiments, the kit can comprise any one of the devices according to the present disclosure.

For example, disclosed herein is a kit comprising: a packed tube comprising: i. a plurality of magnetic beads functionalized to bind a target biological entity; and ii. an aggregation agent, wherein the packed tube is suitable to contain a fluid sample; b. a lid suitable for capping the packed tube, wherein the lid comprises a dispensing tip; and a magnet, wherein the packed tube, the lid, and the magnet are packaged within a common packaging.

In some embodiments, the common packaging can comprise an aluminum bag. In some embodiments, the common packaging can comprise a plastic bag. In some embodiments, the common packaging can comprise paper. In some embodiments, the packaging can comprise a box (e.g., cardboard box). In some embodiments, the common packaging can comprise aluminum bag, plastic bag, box (e.g., cardboard box), paper or a combination thereof. In some embodiments, the common packaging can comprise packaging foams (e.g., expanded polystyrene, PE, PU, cross-linked) to protect items within the packaging. In some embodiment, the common packaging can be compartmentalized to fit items of various shapes (e.g., round, oblong, flat, etc.). In some embodiments, the common packaging can be individual bags or boxes with items collected into a larger box. In some embodiments, common packaging can be made from recyclable materials. In some embodiments, the packaging can be recycled. In some embodiments, the common packaging can be designed to be returnable from the consumer. In some embodiments, the common packaging can be stackable. In some embodiments, the packaging can comprise a desiccant.

In some embodiments, the common packaging can be pharmaceutically acceptable. In some embodiments, the common packaging can be acceptable for research needs. In some embodiments, the common packaging can be suitable for delivery. For example, the common packaging can be suitable for short-or long-range shipping.

In some embodiments, the kit can further comprise a calcium chelation agent or chelator (e.g., EDTA, EGTA, sodium citrate, trisodium citrate, oxalates, BAPTA, BAPTA tetrapotassium salt, BAPTA tetracesium salt, BAPTA tetrasodium salt, BAPTA AM, 5,5′-Dimethyl BAPTA, AM, 5,5′-Difluoro BAPTA, or 5,5′-Dibromo BAPTA) within the common packaging. In some embodiments, the kit can comprise an anticoagulant in the common packaging. In some embodiments, the kit can comprise both anticoagulant and the calcium chelation agent or chelator in the common packaging. In some embodiments, the kit can comprise a protective agent (e.g., aldehyde, oxazolidine, alcohol, cyclic urea, or any combination thereof) to preserve and stabilize cells (e.g., blood cells) in the common packaging. In some embodiments, the protective agent can be a preserving agent (e.g., imidazolidinyl urea or diazolidinyl urea) and an anticoagulant (e.g., K3EDTA).

In some embodiments, the magnetic beads can be functionalized with an antibody and/or capture agents. In some embodiments, the magnetic beads are functionalized to capture albumin, globulin, fibrinogen, hemoglobin, or any combination thereof. In some embodiments, the antibody and/or capture agents functionalized to the magnetic beads can simultaneously bind to multiple biological entities (e.g., blood cell, red blood cells, white blood cells and platelets) or can bind the cells individually.

In some embodiments, the kit can include an aggregation agent used to aggregate a plurality of the target biological entities prior to or in parallel to the capture and separation of the target biological entities from the fluid sample.

In some embodiments, the aggregation agent comprises a polyionic polymer. In some embodiments, a reversing agent capable of cleaving the polyionic polymer can be used to reverse the aggregation can be included in the kit disclosed herein. In some embodiments, the reversing agent can be a chemical compound, composition, or material, either naturally occurring or synthetic, organic, or inorganic, capable of reversing the aggregation of particles by at least partial depolymerization of the polyionic polymer. In some embodiments, the reversing agent can be a buffer or chemical that breaks protein-protein bonds. In some embodiments, that buffer or chemical can be an IgG elution buffer, 100 mM glycine-HCl (pH 2.5-3.0), 100 mM citric acid (pH 3.0), 50-100 mM triethylamine or triethanolamine (pH 11.5), 150 mM ammonium hydroxide (pH 10.5), 0.1 M glycine-NaOH (pH 10.0), gentle Ag/Ab elution buffer, 5 M lithium chloride, 3.5 M magnesium or potassium chloride, 3.0 M potassium chloride, 2.5 M sodium or potassium iodide, 0.2-3.0 M sodium thiocyanate, 0.1 M Tris-acetate with 2.0 M NaCl (pH 7.7), 2-6 M guanidine-HCl, 2-8 M urea, 1.0 M ammonium thiocyanate, 1% sodium deoxycholate, 1% SDS, 10% dioxane, 50% ethylene glycol (pH 8-11.5), or >0.1 M counter ligand or analog.

In some embodiments, the aggregation agent can be an antibody (e.g., polyclonal, or monoclonal antibody) or protein that can bind to a target biological entity. For example, an antibody can bind to CD44, CD45, CD46, CD47, CD29, CD35, and/or CD82, wherein the target entity is a white blood cell, and other antibodies can be selected to bind other known antigens expressed on the biological entity (e.g., on the cell surface). In some embodiments, the aggregation agent comprises a monoclonal, a polyclonal antibody, or combination thereof that can bind to CD44, CD45, CD46, CD47, CD29, CD35, CD82 and/or CD235a.

In some embodiments, the aggregation agent can be an antibody that binds to proteins non-specifically. For example, an antibody can bind to albumin, globulin, hemoglobin and fibrinogen, wherein the sample fluid is venous and/or capillary blood or other biological fluid with these proteins.

In some embodiments, the aggregation agent can be an antibody that binds to the proteins or cellular material in the fibrin clot or platelets, red blood cells, or white blood cells in fibrin clot or any combination thereof. In some embodiments, the aggregation agent can be an antibody that binds the exterior of the clot or the surface of red blood cells, white blood cells, platelets, or any combination thereof. In some embodiments, the magnetic capture and separation of biological entity (e.g., red blood cells, white blood cells, and/or platelets) from fluid sample (e.g., blood) can prevent the formation of fibrin clots without addition of other anticoagulants.

In some embodiments, the aggregation agent can be a clot-activator, human fibrin, and/or an anti-fibrin antibody that can expedite the formation of fibrin clot within the fluid sample.

In some embodiments, the aggregation agent can be an anti-hemoglobin antibody.

In some embodiments, the aggregation agent can be a platelet aggregation inducer (e.g., adenosine diphosphate, collagen, arachidonic acid, thrombin, epinephrine, and/or ristocetin).

In some embodiment, the concentration of aggregation agent used can be at least about 1 mg/mL, at least about 2 mg/mL, at least about 3 mg/mL, at least about 4 mg/mL, at least about 5 mg/mL, at least about 6 mg/mL, at least about 7 mg/mL, at least about 8 mg/mL, at least about 9 mg/mL, or at least about 10 mg/mL. The concentration of aggregation agent used can be about 1 mg/mL to about 6 mg/mL. In some embodiments, the concentration of aggregation agent used can be about 4 mg/mL to 6 mg/mL. In some embodiments, the concentration of aggregation agent used can be about 3 mg/mL to 5 mg/mL. In some embodiments, a solvent used to prepare the aggregation agent can be water or water with buffering salts.

In some embodiments, the plurality of magnetic beads and the aggregation agent can be provided separately or together in a storage buffer.

In some embodiments, the plurality of magnetic beads and the aggregation agent can be mixed prior to adding the fluid sample.

In some embodiments, the plurality of magnetic beads and the aggregation agent can be dried directly in a collection tube or vessel prior to adding the fluid sample.

In some embodiments, the plurality of magnetic beads and the aggregation agent can be lyophilized directly in a tube or vessel prior to adding the fluid sample.

In some embodiments, the plurality of magnetic beads and the aggregation agent can be added to the fluid sample separately, one after the other.

In some embodiments, the plurality of magnetic beads and the aggregation agent can be contained in the tube or vessel of the device disclosed herein (e.g., packed tube), wherein the tube or vessel also holds the fluid sample during the operation of the device.

In some embodiments, the plurality of magnetic beads can be bound to the surface of a functionalized non-magnetic microbead of larger diameter (e.g., 1-fold, 2-fold, 3-fold, 4-fold, 5-fold, 6-fold, 8-fold, 9-fold, or 10-fold larger than that of the magnetic beads) to facilitate the capture of biological entities from fluid samples.

In some embodiments, the magnetic beads can be no larger than 0.25 μm, 0.55 μm, 1 μm, 2 μm, 3 μm, or 4 μm. In some embodiments, the plurality of magnetic beads has a mean greatest diameter of no larger than 4 μm.

In some embodiments, the plurality of magnetic beads can be ferromagnetic, paramagnetic, or superparamagnetic. In some embodiments, the plurality of magnetic beads can be passively absorbed onto the surface of the larger non-magnetic microbead.

In some embodiments, the larger microparticle can be magnetic.

In some embodiments, the captured biological entities can be released from the plurality of magnetic beads for further analysis.

In some embodiments, the tube or vessel or other container can hold the fluid sample, magnetic beads, and aggregation agent. In some embodiments, the tube or vessel or other container can be a packed tube, packed vessel or other packed container comprising the plurality of magnetic beads and the aggregation agent. In some embodiments, the magnetic beads and aggregation agent are lyophilized. In some embodiments, the packed tube comprises lyophilized magnetic beads and aggregation agent. In some embodiments, the tube can be coated in the interior surface with an antibody and/or capture agent that can bind to the surface of the target entity. In some embodiments, the antibody and/or capture agent can be at the bottom half of the tube. In some embodiments, the tube is coated with a hydrophobic material. In some embodiments, the tube is made of a hydrophobic material. In some embodiments, the tube made from a flexible or deformable material (e.g., plastic). In some embodiments, the tube can be made from a flexible or deformable material (e.g., plastic), wherein the tube can be squeezed to dispense supernatant from the top opening of the tube. In some embodiments, the tube can have an inner wall that is functionalized with a hydrophobic coating (e.g., silica, Teflon, fluoropolymers). In some embodiments, the tube can have an inner wall that is functionalized to be non-wettable.

In some embodiments, the kit disclosed herein can comprise a source of a magnetic field. The magnetic field can be introduced to the fluid sample to separate magnetic bead-captured material from non-captured material. The magnetic field can be generated by a single rare earth magnet or electromagnet or any variation thereof. In some embodiments, the source of a magnetic field can be a magnet. In some embodiments, the magnet can be disposed into a bracket, wherein the bracket comprises a receptacle suitable for holding the packed tube in proximity to the magnet. In some embodiments, the magnetic field (e.g., magnet) can be a neodymium magnet.

In some embodiments, the kit can comprise a size-exclusion membrane. In some embodiments, the kit can comprise a filter. In some embodiments, the filter can be coated with magnetic beads, aggregation agents, or any combination of them. In some embodiments, the filter is one layer or a plurality of different layers. In some embodiments, the kit comprises a lid. In some embodiments, the tube lid can include a size-exclusion membrane positioned near the outlet of the lid to capture residual cells in the supernatant. In some embodiments, the lid comprises a size-exclusion membrane in fluid communication with the dispensing tip. In some embodiments, the tube lid can comprise a filter. In some embodiments, the lid further comprises a filter in fluid communication with the dispensing tip. In some embodiments, the filter can be hydrophilic or hydrophobic. In some embodiments, the tube lid can comprise a dispensing tip. In some embodiments, the dispensing tip only dispenses a pre-determined amount of supernatant.

### EXAMPLES

These examples are provided for illustrative purposes only and not to limit the scope of the claims provided herein.

### Example 1: Use of Magnetic Bead-Based Capture of Biological Entity

This example shows how a packed tube (e.g., dropper tube disclosed herein), can be used to efficiently capture a target biological entity, such as blood cells (FIG. 11).

The packed tube comes packed with a plurality of magnetic beads functionalized to bind the target biological entity, and an aggregation agent. First, blood sample (300 μL) from an individual is collected into the packed tube. The blood sample can be obtained by lancing the individual's finger. Next, the packed tube is capped with a lid that has a dispensing tip. The packed tube is agitated by rolling around the tube to mix the blood sample with the magnetic beads and the aggregation agent for 30 seconds. The aggregation agent promotes aggregation of the target biological entity to be captured by the magnetic beads. Next, the packed tube is disposed onto a tube holder that has a magnet at the bottom of the holder. Placing the tube onto the tube holder for 1-4 minutes separates the supernatant or plasma from the magnetic beads. The separated supernatant or plasma is dispensed through the dispensing tip into a collection tube by squeezing the tube.

The plasma is assessed to determine the efficacy of separating the plasma from the target biological entities, such as red blood cells. The plasma is five-fold diluted, collected by using the packed tube, and mixed in a 1:1 ratio with trypan blue stain. Once stained, the plasma is loaded into a hemocytometer and cells are counted under a bright-field microscope. The number of cells counted are used to estimate the purity of the plasma obtained.

### Example 2: Supernatant Yield Using Magnetic Bead-Based Capture of Biological Entity

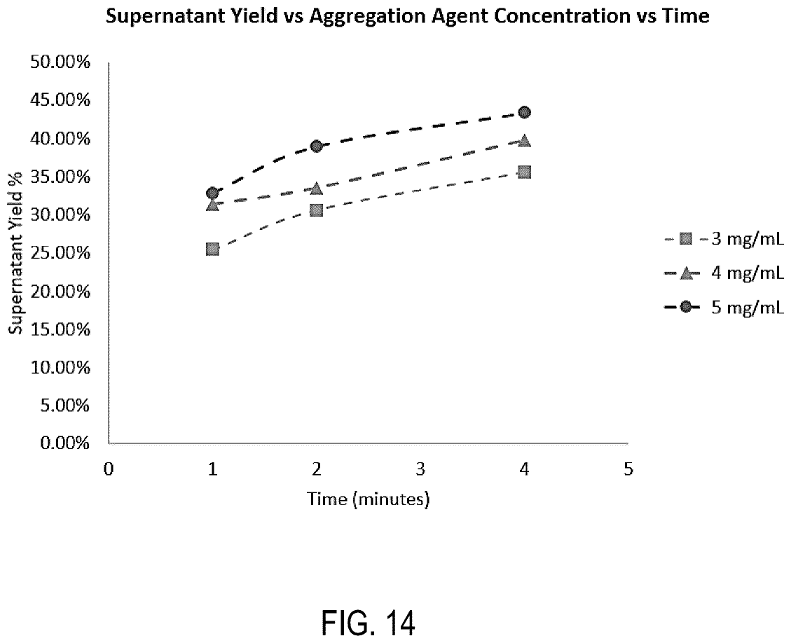

Three aggregation agent concentrations and three incubation times on the magnet were tested according to the method described in Example 1. Percentage of supernatant (i.e., plasma) yield from 300 μL blood was measured (FIG. 14). The concentrations tested were 3 mg/mL, 4 mg/mL, and 5 mg/mL. The solvent used to prepare the aggregation agent concentrations was water or water with buffering salts. The incubation times tested were 1 minute, 2 minutes, and 4 minutes. The percent yield of supernatant for each condition is shown in Table 1.

TABLE 1
Percent yield of supernatant

| | Aggregation Agent Concentration | | | | | |
|---|---|---|---|---|---|---|
| Time on | 3 mg/mL | | 4 mg/mL | | 5 mg/mL | |
| magnet | Percent | | Percent | | Percent | |
| (min) | Yield | STDEV | Yield | STDEV | Yield | STDEV |
| 1 | 25% | 6% | 31% | 8% | 33% | 4% |
| 2 | 31% | 5% | 34% | 10% | 39% | 8% |
| 4 | 36% | 8% | 40% | 3% | 43% | 6% |

What is claimed is:

1. A method of capturing a target biological entity from a fluid sample, the method comprising:
a. obtaining a package wherein the package contains a packed tube, wherein the packed tube is packed with:
i. a plurality of magnetic beads functionalized to bind the target biological entity; and
ii. an aggregation agent, wherein the aggregation agent aggregates the target biological entity when exposed to a plurality of the target biological entities;
b. removing the packed tube from the package;
c. disposing the fluid sample into the packed tube, wherein the fluid sample contains the target biological entity;
d. after the disposing the fluid sample into the packed tube, capping the packed tube with a lid, wherein the lid comprises a dispensing tip;
e. after the capping the packed tube with the lid, agitating the packed tube to mix the fluid sample with the plurality of magnetic beads and the aggregation agent; and
f. after the agitating the packed tube, disposing the packed tube in proximity of a magnetic field, wherein the magnetic field separates the magnetic beads from a supernatant.

2. The method of claim 1, wherein the packed tube comprises an interior surface, wherein the interior surface of the packed tube is coated at least in part with a chelation agent.

3. The method of claim 1, wherein the packed tube contains a chelation agent.

4. The method of claim 1, wherein the method further comprises: g. dispensing the supernatant through the dispensing tip into a collection tube.

5. The method of claim 4, wherein the method further comprises: f. squeezing the packed tube to dispense the supernatant.

6. The method of claim 1, wherein the packed tube comprises an interior surface, wherein the interior surface of the packed tube is coated at least in part with an anticoagulant.

7. The method of claim 1, wherein the packed tube contains an anticoagulant.

8. The method of claim 1, wherein the fluid sample is blood.

9. The method of claim 1, wherein the target biological entity is a blood cell.

10. The method of claim 1, wherein the packed tube is made of a deformable material.

11. The method of claim 1, wherein the packed tube is coated with a hydrophobic material.

12. The method of claim 1, wherein the plurality of magnetic beads has a mean greatest diameter of no larger than 4 μm.

13. The method of claim 1, wherein the aggregation agent is an antibody.

14. The method of claim 1, wherein the aggregation agent is present at a concentration from about 3 mg/mL to about 5 mg/mL in water.

15. The method of claim 1, wherein the plurality of magnetic beads and the aggregation agent are lyophilized.

16. The method of claim 1, wherein the supernatant is substantially free of the target biological entity.

17. The method of claim 1, wherein the lid further comprises a size-exclusion membrane in fluid communication with the dispensing tip.

18. The method of claim 1, wherein the lid further comprises a filter in fluid communication with the dispensing tip.

19. The method of claim 1, wherein the magnetic beads are substantially separated from the supernatant within one minute of disposing the packed tube in proximity of the magnetic field.

20. The method of claim 1, wherein at least about 95% of the target biological entity is separated from the supernatant.
